(19) United States
(12) Patent Application Publication
(10) Pub. No.: US 2011/0207173 A1
(43) Pub. Date: Aug. 25, 2011

(54) Self-Replicating Vector Lacking An Antibiotic-Resistance Gene

(75) Inventors: Regis Sodoyer, Saint Genis les Ollieres (FR); Denis Speck, Sainte Foy les Lyon (FR); Isabelle Peubez, Saint Pierre la Palud (FR); Nicolas Chaudet, Soucieu en Jarrest (FR)

(73) Assignee: SANOFI PASTEUR S.A., Lyon (FR)

(21) Appl. No.: 13/054,427

(22) PCT Filed: Jul. 9, 2009
(86) PCT No.: PCT/FR2009/000851

§ 371 Date: Jan. 14, 2011

Related U.S. Application Data
(60) Provisional application No. 61/104,524, filed on Oct. 10, 2008.

(30) Foreign Application Priority Data
Jul. 18, 2008 (FR) 0854887

(51) Int. Cl. C12P 21/00; C12N 15/63; C12N 1/00; C12N 1/21; C12N 15/74

(52) U.S. Cl. 435/69.3; 435/320.1; 435/243; 435/252.33; 435/69.1; 435/91.4

(57) ABSTRACT

The subject matter of the present invention is a novel vector and the use thereof for producing a heterologous protein or a gene of interest, that can be used, for example, in the context of an immunization or gene therapy programme and concerns in particular a self-replicating vector lacking an antibiotic-resistance gene, comprising a sequence encoding the ccdA protein functionally linked to a first promoter, the sequence of the Cer locus and a heterologous sequence, functionally linked to a second promoter.

The present invention relates to a new vector and to the use thereof for the production of a heterologous protein or of a gene of interest that can be used, for example, in the context of an immunization or gene therapy program.

The vectors comprise at least one selectable marker, the presence of which is necessary during their construction process and also during the cell culture phase which results in their amplification and optionally in the production of a protein of interest.

The selectable markers conventionally used are antibiotic-resistance genes.

The risks potentially associated with the use of these resistance genes, such as dissemination in the environment or transfer of the gene to a pathogenic strain, have led health authorities to limit the use of the latter. The presence of a gene for resistance to an antibiotic is today considered to be a major drawback for use in humans.

Several alternative solutions have therefore been proposed. Mention may, for example, be made of selection systems based on the complementation of an essential gene. These systems all have the drawback of requiring the construction of a specific strain deficient in said essential gene and the obligatory use of defined media which do not contain the product of the essential gene.

The objective of the present invention is to provide a new vector which can be used on an industrial scale, which has the double advantage of producing a high expression yield, this being the case in the absence of any use of antibiotics, and which can therefore be used for large volumes (for example, 1000-10 000 liters).

This system is therefore particularly advantageous in the context of an industrial production of components that can be used in humans.

The present invention therefore provides a self-replicating vector devoid of any antibiotic-resistance gene, comprising:
a sequence encoding the ccdA protein functionally linked to a first promoter,
the sequence of the Cer locus, and
a heterologous sequence functionally linked to a second promoter.

According to one particular embodiment, the first promoter is the mob constitutive promoter.

According to another embodiment, the second promoter is an inducible promoter, in particular the second promoter is the T7 promoter.

According to one particular embodiment, the heterologous sequence encodes a vaccine antigen.

According to another particular embodiment, the heterologous sequence corresponds to a sequence that can be used in the context of a gene therapy.

According to yet another embodiment, the heterologous sequence encodes an enzyme.

According to another aspect, the present invention relates to a prokaryotic cell expressing the ccdB protein, comprising a vector as defined above.

According to one particular aspect, said prokaryotic cell is an E. coli cell.

According to another aspect, the present invention relates to a method for producing a heterologous protein, comprising the steps of:

(a) inoculating an appropriate culture medium with prokaryotic cells expressing the ccdB protein and containing a vector as defined above,

(b) fermenter culturing the cell thus transformed in the absence of antibiotic, and

(c) recovering the heterologous protein produced during step (b) from the supernatant or from the cell pellet.

According to one particular embodiment, the present invention relates to a method for producing rEPA.

According to another aspect, the present invention relates to a method for producing a self-replicating vector as defined above, comprising the steps of

(a) inoculating an appropriate culture medium with prokaryotic cells expressing the ccdB protein and containing a vector as defined above,

(b) fermenter culturing the cell thus transformed in the absence of antibiotic, and

(c) recovering the vector produced during step (b).

According to another aspect, the present invention relates to a method for constructing a self-replicating vector as defined above, comprising the steps of:

(a) constructing a self-replicating vector comprising a functional antibiotic-resistance gene flanked respectively by a sequence 1 and a sequence 2, in which the sequences 1 and 2 are two overlapping sequences of the sequence encoding the ccdA protein, which, after homologous recombination, reconstitutes a functional ccdA sequence,

(b) linearizing said vector by using a restriction enzyme which cleaves the vector only between the sequences 1 and 2,

(c) transforming a prokaryotic cell expressing the ccdB protein, and

(d) recovering the prokaryotic cells comprising the self-replicating vector.

The invention will be described in detail in the description which follows, with reference to the figures attached in the annex, in which:

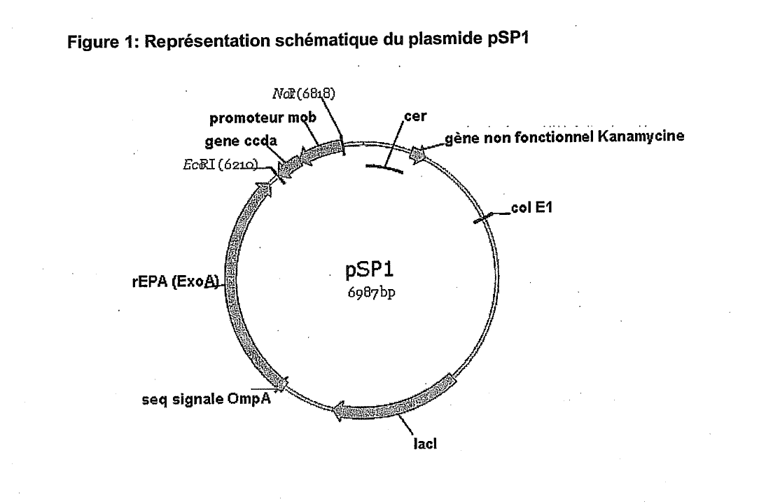

FIG. 1 is a schematic representation of the plasmid pSP1.

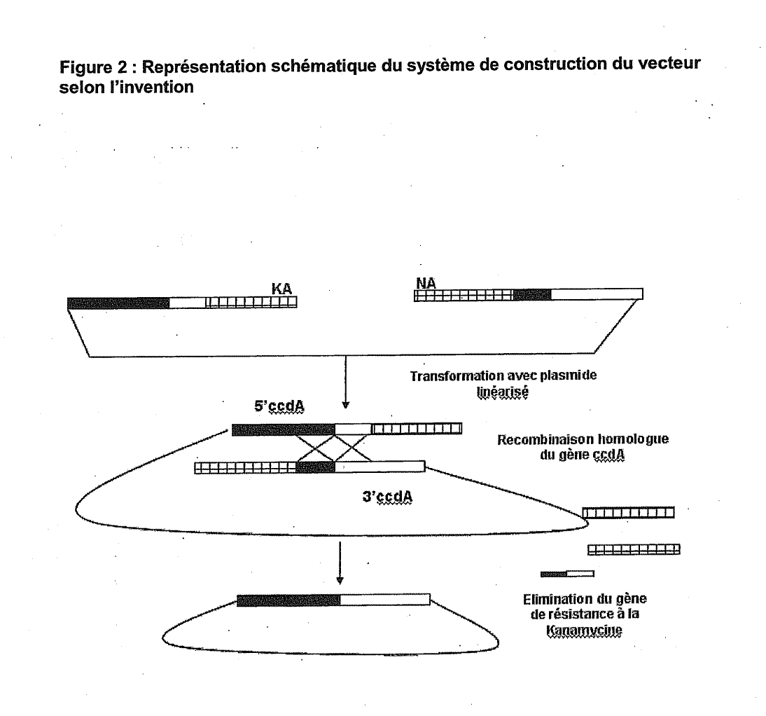

FIG. 2 is a schematic representation of the method for constructing a vector according to the invention.

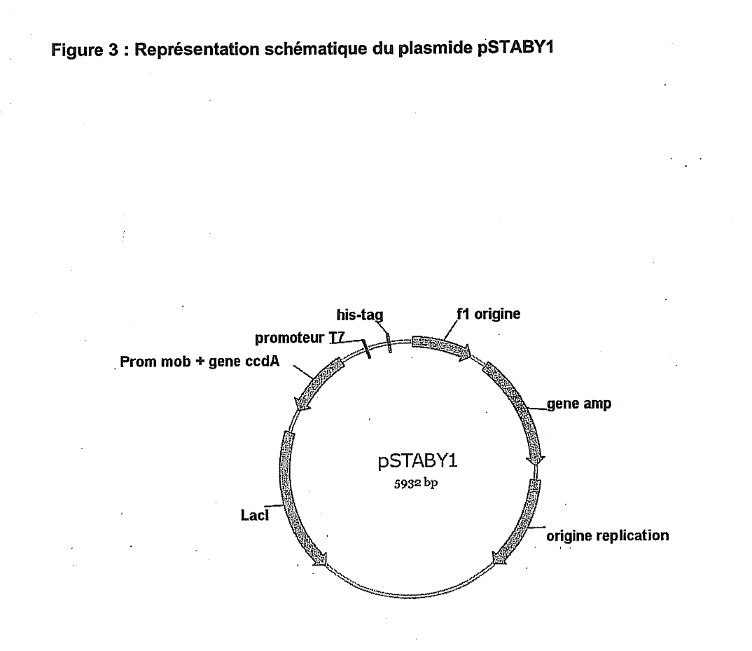

FIG. 3 is a schematic representation of the plasmid pSTABY1.

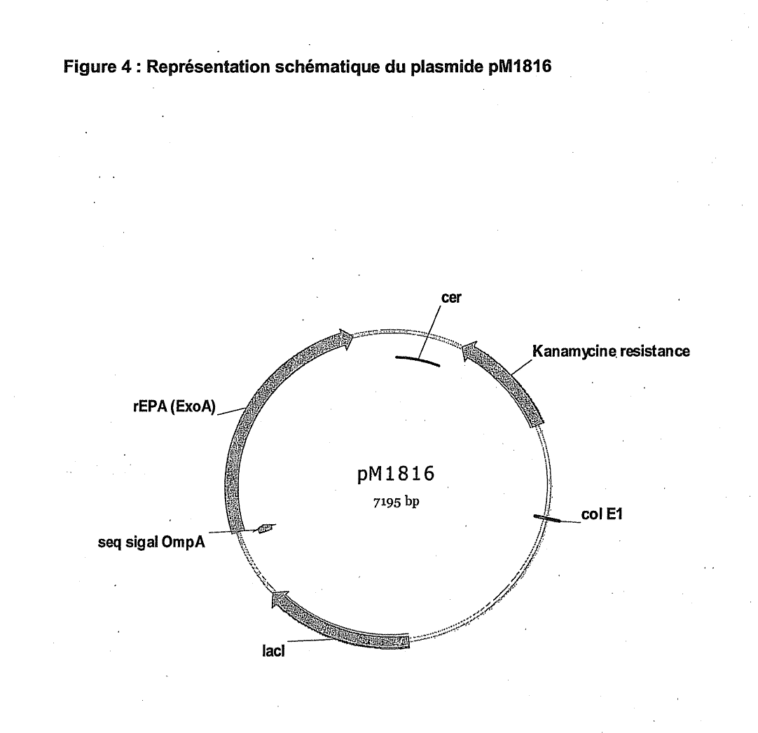

FIG. 4 is a schematic representation of the plasmid PM1816.

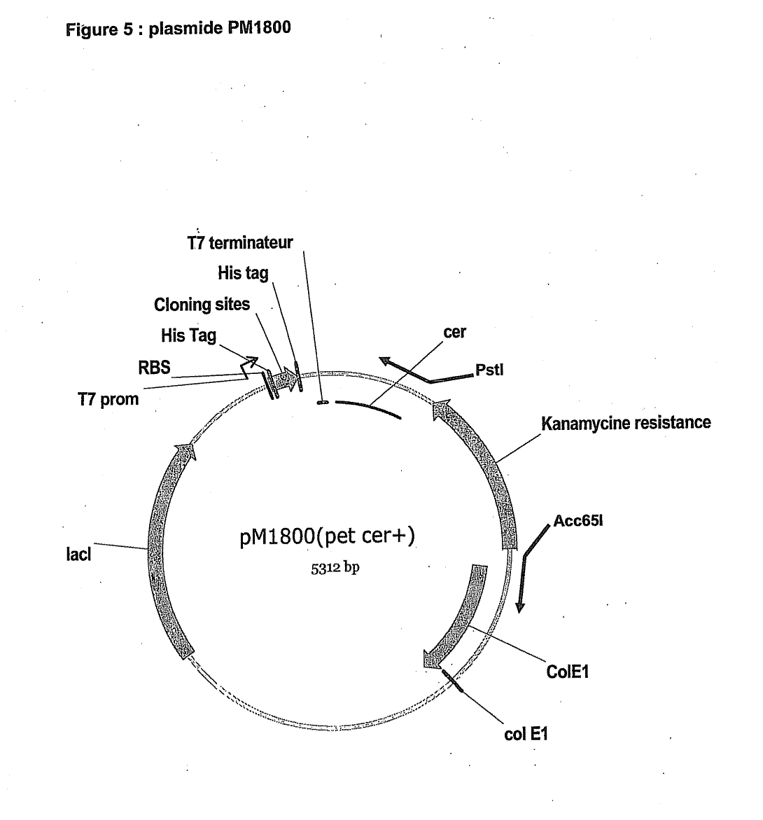

FIG. 5 is a schematic representation of the plasmid PM1800.

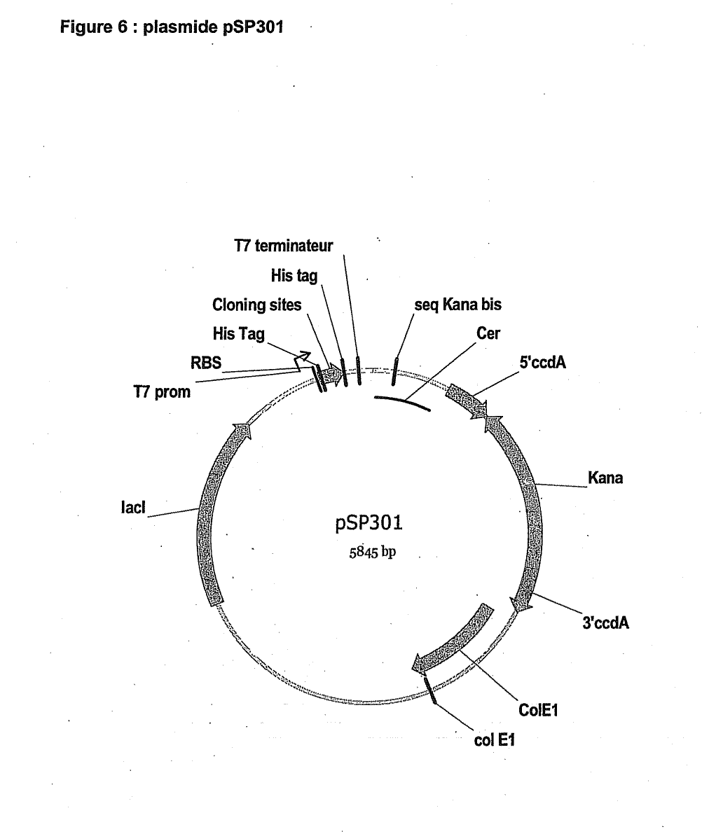

FIG. 6 is a schematic representation of the plasmid pSP301.

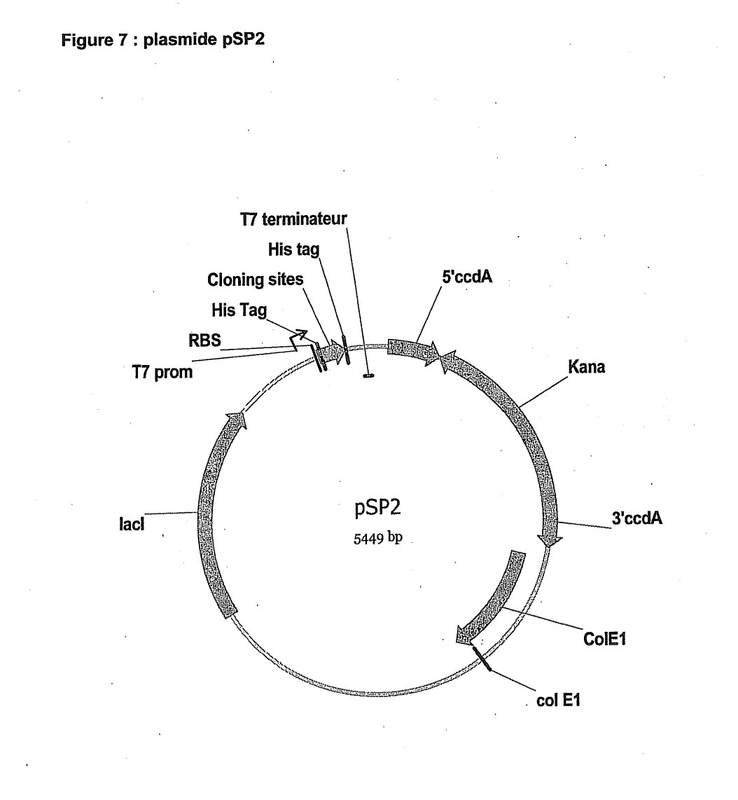

FIG. 7 is a schematic representation of the plasmid pSP2.

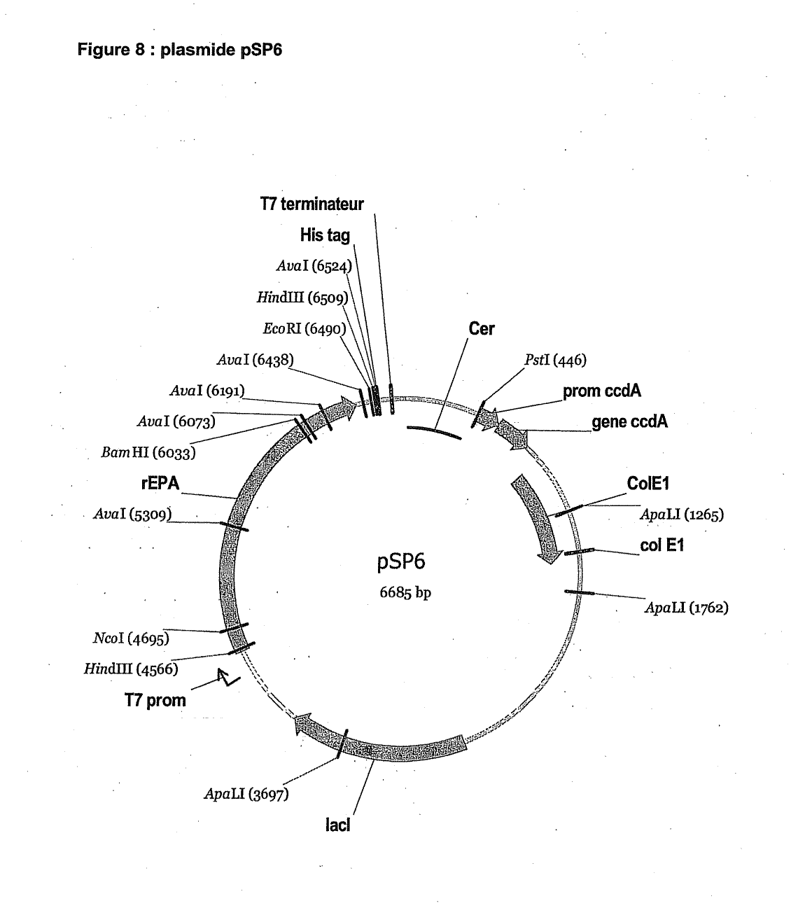

FIG. 8 is a schematic representation of the plasmid pSP6.

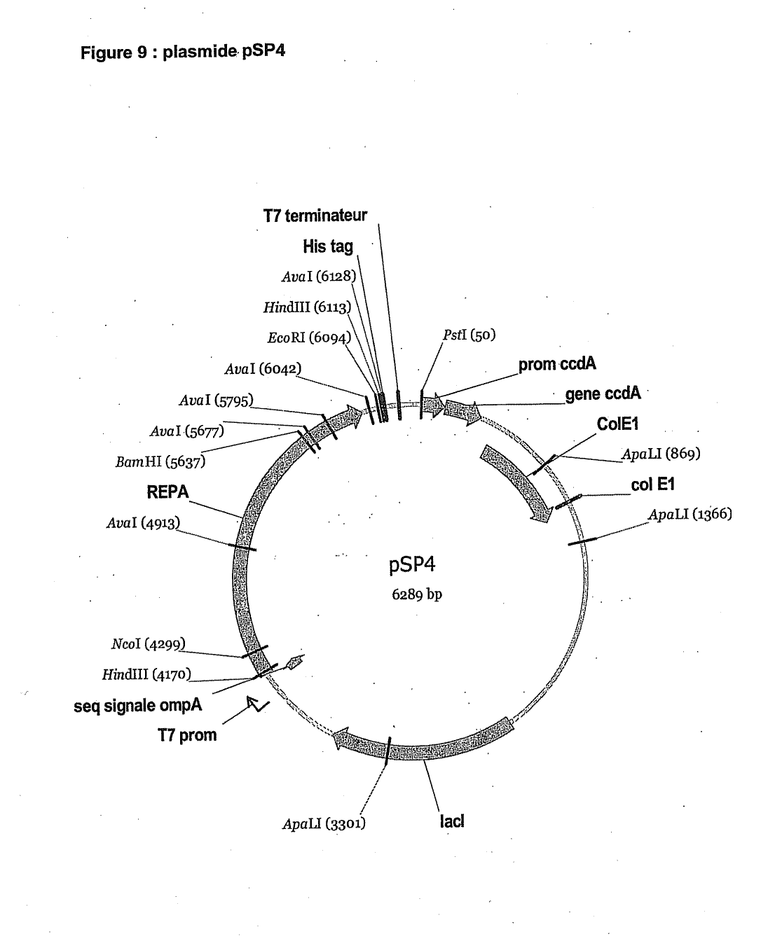

FIG. 9 is a schematic representation of the plasmid pSP4.

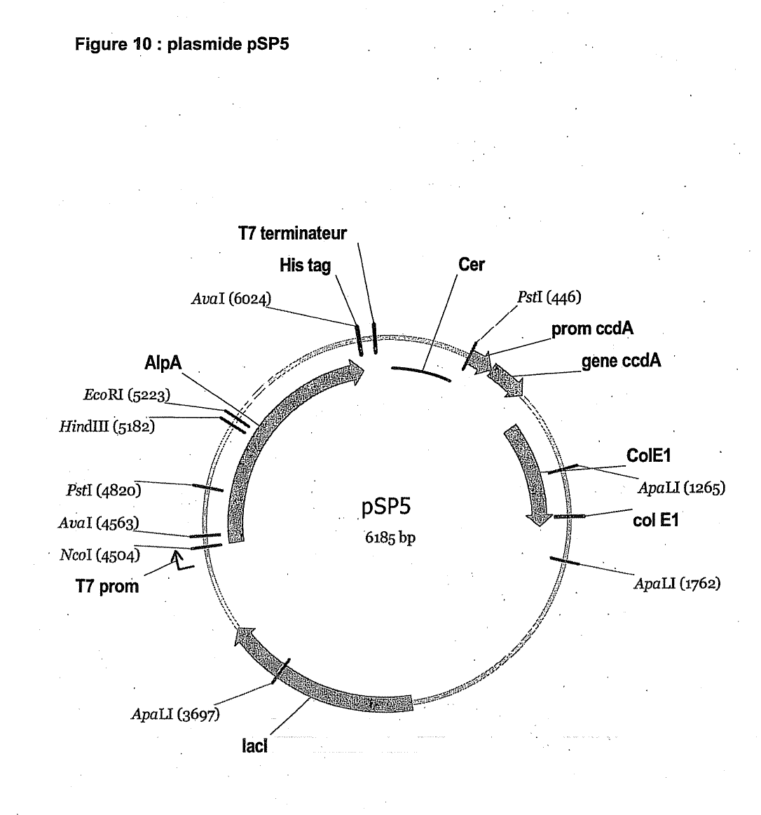

FIG. 10 is a schematic representation of the plasmid pSP5.

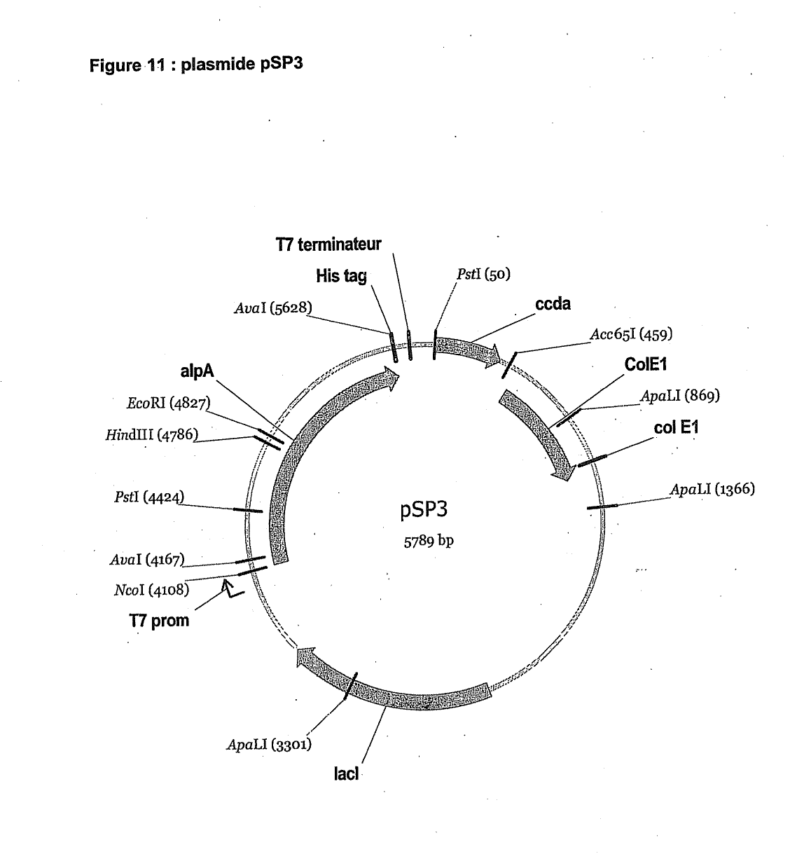

FIG. 11 is a schematic representation of the plasmid pSP3.

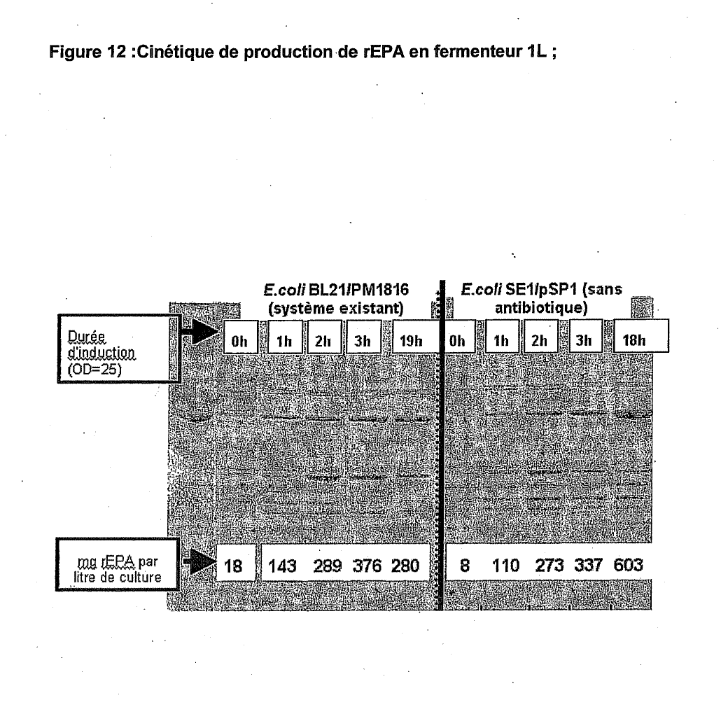

FIG. 12 gives the kinetics for production of rEPA in a 1-liter fermenter.

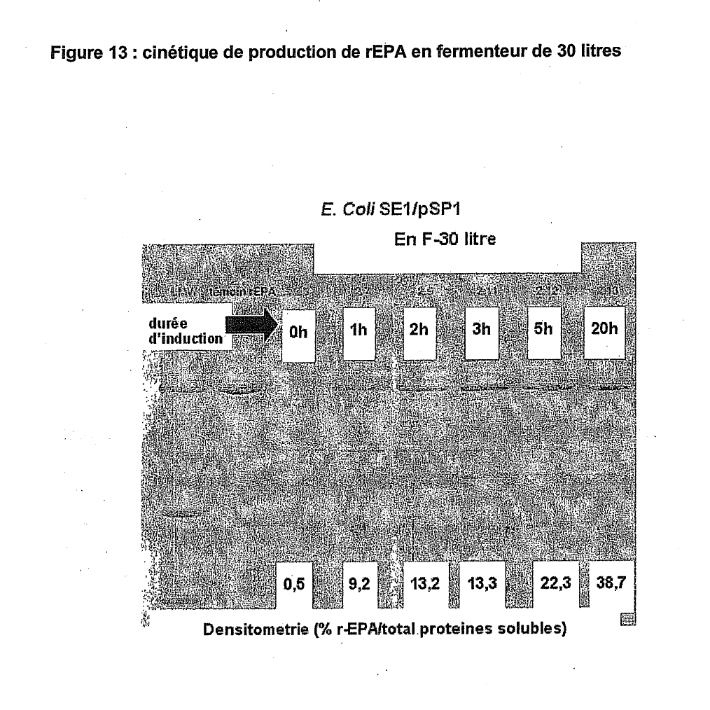

FIG. 13 gives the kinetics for production of rEPA in a 30-liter fermenter.

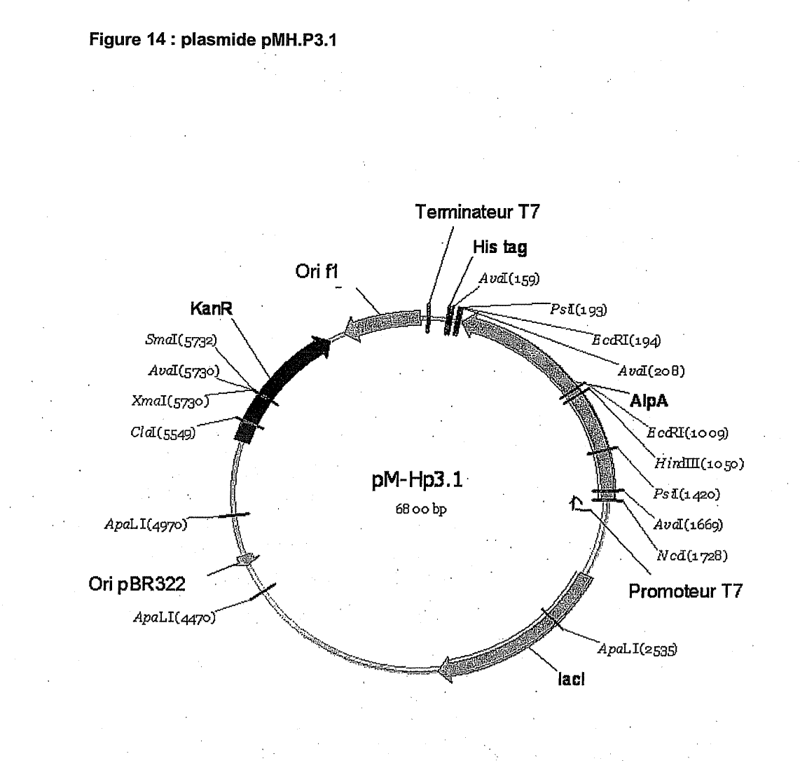

FIG. 14 gives a schematic representation of the plasmid pMH.P3.1

According to a first aspect, the present invention therefore relates to a self-replicating vector comprising:
a sequence encoding the ccdA protein, functionally linked to a first promoter,
the sequence of the Cer locus, and
a heterologous sequence functionally linked to a second promoter.

The term “self-replicating vector” is intended to mean a nucleic acid molecule capable of replicating autonomously in a host cell. The vector according to the invention may be a plasmid, a phagemid or a bacteriophage. A self-replicating vector therefore comprises one or more sequences directing or controlling the expression of the product of the nucleic acid sequences contained in said vector. Such a vector therefore comprises in particular an origin of replication that is functional in the host cell transformed with said vector. The vector according to the invention is advantageously a plasmid, in particular a plasmid capable of replicating in an E. coli cell.

Any origin of replication conventionally used in self-replicating vectors may be used in the context of the present invention. The origin of replication confers a more or less high specificity with regard to the host cell and conditions the number of copies of said vector. The origin that may be used may be a single-copy origin, a low-copy-number origin or a high-copy-number origin. In the context of the use of the vector for the expression of a protein of interest or for the production of vectors that can be used for DNA immunization or gene therapy, the origin of replication is advantageously a high-copy-number origin (conventionally understood to mean several hundred copies) such as colE1.

The self-replicating vectors according to the invention are vectors devoid of antibiotic-resistance genes.

In the context of the present invention, the expression “vector devoid of antibiotic resistance gene” is intended to mean a vector which does not contain any antibiotic-resistance gene or which comprises all or part of a nonfunctional antibiotic-resistance gene. Advantageously, the vector according to the invention does not comprise any antibiotic-resistance gene.

The vector according to the invention comprises as single selectable marker, a sequence encoding a ccdA protein. The ccdA protein functions in association with a host cell comprising a functional gene encoding the ccdB protein.

The gene encoding the poison (ccdB) is introduced into the bacterial chromosome of the host cell. It encodes a stable protein of approximately 100 amino acids, which binds to gyrase, inducing death of the bacterium.

The gene encoding the antidote (ccdA), for its part, encodes an unstable protein of approximately 90 amino acids, which neutralizes the poison protein. This ccdA gene is introduced into the vector according to the invention under the control of a constitutive promoter.

The expression of the poison gene is under the control of a promoter which can be strongly repressed by the antidote or the poison-antidote complex. Consequently, when the vector is present in the host cell, the poison is not produced. Moreover, when the vector is lost, the antidote is degraded by a protease and the induced poison production causes death of the host cell. This ccdA/ccdB selection system is described in detail by Szpirer CY and Milinkovitch MC in Biotechniques. 2005 May; 38(5):775-81.

Reference may also be made to document WO 99/58652 for a detailed description of this selection system.

Said sequence encoding the ccdA protein is functionally linked to a first promoter. Any constitutive promoter conventionally used in vectors may be used in the context of the present invention. A noninducible promoter will advantageously be used. It is not necessary to use a strong promoter. Furthermore, the promoter does not necessarily have to be repressed by ccdB or the ccdB/A complex. Advantageously, the mob constitutive promoter is used.

The vector according to the invention also comprises the sequence of the Cer locus. Said sequence can be inserted into the vector in any orientation and at any position. Said Cer sequence is described by Summers D. K. and Sherratt, D. J. in EMBO J. 7 (3), 851-858 (1988). Said Cer sequence is reproduced in the sequence listing in SEQ ID No. 1.

The vector according to the invention also comprises a heterologous sequence functionally linked to a second promoter.

In the context of the present invention, the “heterologous sequence” is intended to mean a sequence encoding a therapeutic protein or a protein that can be used for diagnostic purposes or encoding a vaccine antigen, and also encoding any protein of commercial or industrial interest or a sequence of a heterologous gene that is of interest in DNA vaccination or gene therapy.

By way of examples of therapeutic proteins, mention may in particular be made of: blood derivatives, hormones, in particular growth hormone, lymphokines, proteins encoding an enzyme activity capable of converting a prodrug to a drug, in particular in the context of a cancer treatment protocol, as described, for example, in Table 3 of the general review by Kratz et al. ChemMedChem. 2008 January; 3(1):20-53.

By way of examples of a vaccine antigen, mention may be made of proteins that can be used in an immunization program and also genes that can be used in the context of a

DNA vaccination, in particular surface proteins of a bacterial pathogen but also viral envelope proteins, parasite proteins or alternatively surface markers of tumor cells.

By way of example of sequences that can be used in the context of a gene therapy, mention may be made of the sequences that can be used in the context of the treatment of genetic diseases such as cystic fibrosis.

A heterologous sequence encoding an enzyme, for example an enzyme of industrial interest such as benzonase, trypsin or alternatively a molecule capable of intervening in a biocatalytic process can be inserted into a self-replicating vector according to the invention.

More specifically, mention may be made, by way of nonlimiting example of a vaccine antigen that can be produced with the vector according to the invention, of: Pseudomonas aeruginosa exoprotein A detoxified, for example, by deletion of the Glu 553 residue (rEPA), the tbpB (transferin binding protein) antigen of N. meningitidis B (Legrain et al. Protein Expr Purif. 1995 October; 6(5):570-8), the Helicobacter pilori AIpA protein, influenza virus hemaglutinin HA, such as, for example, the sequence SEQ ID No. 5 or the corresponding sequences derived from other flu virus strains, the sequence encoding CFTR, such as, for example, SEQ ID No. 6, advantageously without introns (Babenko, A. P. J. Biol. Chem. 283 (14), 8778-8782 (2008)), the antigens originating from the “sporozoite” form of Plasmodium falciparum, (such as the major sporozoite surface protein (circumsporozoite protein), LSA3 or the Pfs 16 antigen), and also antigens originating from the “merozoite” form of Plasmodium falciparum (such as the MSP1, MSP2, MSP3, EBA-175, Rhop-1, Rhop-2, Rhop-3, RAP-1, RAP-2, RAP-3, Pf155/RESA or AMA-1 antigen). The sequences encoding these proteins are known and available on various databases. For example, the complete sequence of the LSA 3 gene is 12240 base pairs long and encodes a protein of 1558 amino acids. The nucleotide and protein sequences are described in the EMBL data bank under accession numbers AE001424 and uniprot 096275-PLAF7. As heterologous sequence, use may be made of the sequence encoding the whole protein for fragments of this protein, such as those described in WO 02/38176. Use is customarily made of the whole protein (which may contain one or more point mutations so as to take into account the variations which exist between the strains of Plasmodium falciparum) or a fragment of this protein of which the amino acid sequence has at least 80%, in particular at least 90%, especially at least 95% to 99% identity relative to the whole sequence described in uniprot 096275-PLAF7.

The system according to the invention is particularly suitable for the production of proteins for which glycosylation is absent or not essential for their effectiveness or for the induction of an immune response; on the other hand, sequences encoding glycosylated proteins may be used, for example, in DNA vaccination.

Any constitutive or inducible promoter conventionally used in vectors can be used in the context of the present invention as second promoter. By way of example, mention may be made of the T7, T5, arabinose, lac, Trp promoters or any other promoter derived from microorganisms capable of functioning in a prokaryotic host cell, in particular Escherichia coli. In the case of vectors used for gene therapy or DNA immunization, the expression of the gene of interest may be under the control of a promoter which functions in eukaryotic cells, such as the CMV promoter or the SV40 promoter or alternatively promoters having a cell specificity.

The vector according to the invention may also contain expression-regulating sequences such as, for example, a transcription-regulating terminator sequence, a signal sequence which allows the exportation of the expressed protein to the periplasmic compartment of the host cell or the secretion of said protein into the culture supernatant of the host cells, and also a multiple cloning site. These sequences are well known to those skilled in the art.

The vector according to the invention may be constructed by any conventional genetic engineering technique.

Although use of the ccdA/ccdB selection is possible from the first steps of construction of the vector, it is possible to maintain an antibiotic-mediated selection pressure up to the final phase of the vector construction method. The elimination of the antibiotic-resistance gene can subsequently be carried out by simple digestion with a restriction enzyme at unique sites, flanking the antibiotic-resistance gene, and then religation of the vector on itself.

The final host cell/vector pair is obtained after transformation of the prokaryotic cell with the plasmid that has been religated on itself. A means of verifying the elimination of said gene then consists in subculturing the colonies obtained, after transformation, in parallel, on dishes containing or not containing the antibiotic, or alternatively in verifying the absence of said gene by PCR, restriction mapping or any other appropriate method. In any event, this additional verification proves to be tedious.

The inventors have demonstrated another system for constructing the vector according to the invention, which has the advantage of resulting in easy selection of the clones sought.

According to a second aspect, a subject of the present invention is therefore a method for producing a self-replicating vector as defined above, comprising the steps of:

(a) constructing a self-replicating vector comprising an antibiotic-resistance gene flanked, respectively, by a sequence 1 and a sequence 2, in which the sequences 1 and 2 are two overlapping sequences of the ccdA sequence, which, after homologous recombination, reconstitutes a functional ccdA sequence,

(b) linearizing said vector by using a restriction enzyme having a restriction site only between the sequences 1 and 2,

(c) transforming a prokaryotic cell expressing a functional ccdA protein, and

(d) recovering the cells containing the vector according to the invention.

The new method according to the invention uses a recombination event which makes it possible, in a one and only step, to eliminate the antibiotic-resistance gene and to assemble a ccdA gene in its functional form. In summary, the ccdA gene is cloned in the form of 2 separate and individually nonfunctional elements located on either side of the antibiotic-resistance gene. The parts referred to as 5′ and 3′ of ccdA are defined in such a way as to contain a common sequence of, for example, 200 nucleotides in length. As long as the antibiotic-resistance gene is present, the ccdA assembly is nonfunctional. After digestion with a restriction enzyme having a site located only between the sequences 1 and 2, for example within the antibiotic-resistance gene, a linear DNA fragment is obtained. Through transfection of prokaryotic cells, for example of competent E. coli cells, this DNA molecule can recircularize by homologous recombination of the overlapping ccdA fragments. This step makes it possible, on the one hand, to eliminate the antibiotic-resistance gene and, on the other hand, to select the clones containing the vector according to the invention, insofar as the ccdA gene can be functional only after elimination of the antibiotic-resistance gene. A schematic representation of the method according to the invention is given in FIG. 2.

For the implementation of the method above, reference may be made to a reference book in terms of genetic engineering, such as Maniatis et al. “Condensed Protocols from Molecular Cloning: A Laboratory Manual” by Joseph Sambrook and David W. Russell, which describes all the operating conditions to be used for the steps for construction of a vector, linearization, transformation and recovery of the clones. The self-replicating vector can subsequently be readily recovered from the transformed cells by any conventional method well known to those skilled in the art.

As regards in particular the homologous recombination, the size of the overlapping sequence can vary to a large extent. Sequences which overlap over only 5 nucleotides can be used. The only important element in the selection of the sequences 1 and 2 is that the two sequences, after homologous recombination, reconstitute a functional ccdA sequence, for example the complete ccdA sequence. Furthermore, they should flank the antibiotic-resistance gene so as to allow elimination of the latter.

Any self-replicating vector as defined above can be advantageously produced by the new method according to the invention.

According to another aspect, the present invention therefore relates to a prokaryotic cell containing a self-replicating vector as defined above.

Any prokaryotic cell expressing a functional ccdB protein can be used in the context of the present invention. A ccdB protein is functional if it is capable of significantly inhibiting the action of gyrase, i.e. if it is capable of inducing a lethal effect on the prokaryotic cell. It is therefore possible to use the whole ccdB protein or fragments thereof which are capable of bringing about cell death in the absence of ccdA protein. An E. coli cell expressing the ccdB protein, in particular an E. coli cell in which the sequence encoding the ccdB protein has been inserted into the bacterial genome, will advantageously be used. The insertion of the ccdB sequence into the bacterial genome can be carried out by any method well known to those skilled in the art for performing such an insertion.

According to another aspect, the present invention therefore relates to a method for producing a heterologous protein, comprising the steps of:

(a) inoculating an appropriate culture medium with prokaryotic cells expressing the ccdB protein as defined above and containing a vector as defined above,

(b) in a fermenter, culturing in the absence of antibiotic, and

(c) recovering the heterologous protein produced during step (b) from the supernatant or from the cell pellet.

A subject of the present invention is also a method for producing a self-replicating vector as defined above, comprising the steps of:

(a) inoculating an appropriate culture medium with prokaryotic cells expressing the ccdB protein as defined above and containing a vector as defined above,

(b) fermenter culturing the cell thus transformed in the absence of antibiotic, and

(c) recovering the vector produced during step (b).

Conventionally, to implement these methods, the prokaryotic cells, advantageously E. coli cells, originate from a freeze-dried material or a frozen material; they are inoculated into a culture medium volume generally not exceeding 1 liter. After an overnight period of culture or when the optical density of the medium is sufficient, this first culture is transferred into a second culture medium which is identical to or different than the first, but the volume of which may be at least 10, in particular at least 20 and especially at least 30 times or 60 times greater. This second culture, inoculated between 1% and 10%, is carried out in a fermenter of 1 to 10 000 liters, in particular in a fermenter of 30 to 10 000 liters, especially in a fermenter of 30 to 500 liters, such as, for example, 50 to 100 liters.

In the case of the production of products of commercial interest, such as enzymes, fermenters of larger volumes may be used. Fermenters of 1000 to 100 000 liters, for example 60 000 liters, may be used for this last culture step. During the culture period, which is for example from 6 h to 24 h, a temperature of the order of 25° C. to 42° C., in general from 30 to 37° C., a pH of 6.5 and 7.5, shaking between 200 and 1500 rpm, a pressure of 0 to 500 mb, a pO₂ adjusted between 20% and 50% and an equivalent air flow rate of 1 to 2 volumes of air per volume of medium and per minute, which can be enriched in oxygen representing 0 to 50% of the total gas flow, are customarily used.

Any culture medium suitable for the growth of the prokaryotic cell used may be employed. A large number of culture media are described in the literature and are commercially available. By way of example, mention may be made of the E. coli cell culture media used in the context of the present invention.

At the end of the exponential cell growth phase, it is possible to further amplify the biomass by transferring it into another fermenter of greater capacity using the same procedure. The culture volumes can reach or even exceed 10 000 liters. The fermenter culturing step is generally carried out according to the “batch” mode. It is also possible to adopt other fermenter culturing modes such as the “fed batch” mode. In such a situation, a nutritive supplement comprising carbohydrates is added to the medium during the exponential growth phase so as to sustain the bacterial multiplication and to obtain a higher cell density at the end of the exponential phase. The amount of carbohydrate added is evaluated according to the cell density and the cell growth rate. The fed batch culturing mode is particularly advantageous in the context of the present invention.

In the case of the production of a protein of interest, at the end of culturing, if the protein of interest is secreted into the supernatant, said supernatant is removed and the protein is purified. If the protein is not secreted into the supernatant, the cell pellet is recovered and the protein of interest is extracted and then purified from said cell pellet. Any method described in the literature as being suitable for this purpose can be used in the context of the present invention. Reference may, for example, be made to the book “Protein purification” 2^{nd} edition, Janson, J-C & Ryden, L, 1998.

According to one particular embodiment, the prokaryotic strain used is a strain of E. coli expressing the ccdB protein.

The method is advantageously used for the production of the proteins identified above, in particular for the production of rEPA.

In the case of the production of a vector according to the invention, the vector is recovered from the cell pellet and purified. Any known method conventionally used in the literature for this purpose can be used in the context of the present invention.

The inventors have demonstrated that the new vector according to the invention results in a significant improvement in the amount of proteins of interest or of vector of interest produced. The vector according to the invention results in an improved containment in the prokaryotic cell expressing the ccdB protein.

The containment of the vector in a prokaryotic cell can be readily evaluated, for example, by determination, at a time t of the culture, of the proportion of cells no longer containing the vector. Conventionally, this determination is carried out by PCR evaluation of the presence of vector DNA and counting.

The expression “improved containment in the prokaryotic cell” is intended to mean an at least 20% decrease in prokaryotic cells having lost the vector, by comparison with a vector/prokaryotic cell system in which the ccdA/Cer system is replaced with a conventional selection system using an antibiotic-resistance gene, under the same culture conditions after a culture period of at least 18 hours and using the same method of determination. The decrease measured is advantageously at least 30%, in particular at least 50%, especially at least 70%.

The vector according to the invention results in an improved production, after fermenter culturing, of proteins of interest or vectors of interest.

The expression “improvement in the fermenter production” is intended to mean an at least 10% increase in the amount of protein of interest produced relative to the amount of total proteins, as determined, after fermenter culturing for 18 h, by densitometric analysis of an electrophoresis gel stained either with coomassie blue or with silver nitrate, by comparison with a production carried out under the same conditions but using an antibiotic-resistance gene as sole selection system (without cer fragment). Such a test is described in the examples which follow.

When one is interested in the production of the vector as such, for example for DNA vaccination purposes, the expression “improvement in the fermenter production” is intended to mean an at least 10% by weight increase in the amount of vector DNA, as determined, after 18 h of fermenter culturing of the host strain containing the vector of interest, by quantitative DNA analysis by measurement at 260 nm on a purified vector sample, by comparison with a production carried out under the same conditions and quantitatively analyzed under the same conditions but using an antibiotic-resistance gene as sole selection system (without cer fragment).

### EXAMPLE 1

### Construction of a Vector According to the Invention Expressing the rEPA Protein

First of all, the mob promoter and the ccdA gene (SEQ ID No. 7) were amplified by PCR with the primers NotccdA+(SEQ ID No. 2) and EcoRIccdA-(SEQ ID No. 3) from the plasmid pStaby1 (5932 by sold by the company Delphi genetics) used as template under the following conditions:

100 ng plasmid pStaby1; 10 μl×10 “high fidelity” buffer (Invitrogen); 125 μM dNTP mix; 1 μM 5′-primer; 1 μM 3′-primer; 3 μl 50 mM MgSO₄; 2.5 U “high fidelity” Taq polymerase (Invitrogen); H₂O qs 100 μl using the PCR program below.

PCR Program

| Step | Temperature | Time | Number of cycles |
|---|---|---|---|
| Denaturation | 97° C. | 30 sec | 30 |
| Hybridization | 56° C. | 1 min | |
| Polymerization | 72° C. | 30 sec | |
| Polymerization | 72° C. | 7 min | 1 |

The DNA fragments are purified by preparative 1% agarose gel electrophoresis in 1× Tris-acetate-EDTA buffer. The bands corresponding to the fragments of interest are cut out and the DNA is recovered by electroelution and purified with the Qiaquik kit from the company Qiagen.

The ccdA PCR fragment is subsequently digested with 45 U of NotI and 30 U of EcoRI per 2250 ng of DNA. In parallel, 5 μg of plasmid PM1816 (7195 bp) is digested with 30 U of NotI and 20 U of EcoRI enzyme. The restriction enzymes from Invitrogen or from New England Biolabs are used with the corresponding X10 buffers sold by the supplier. The reactions are carried out for 2 h at 37° C.

The plasmid PM1816 was constructed from the plasmid pET28c (Novagen) which was amplified by PCR so as to eliminate the F1 origin. This amplification made it possible to create the PacI and AscI sites, and the Cer fragment was then cloned between these same restriction sites. The vector thus created was named pM1800 (cf. FIG. 5 for a schematic representation). Finally, the sequence SEQ ID No. 9 (containing RBS-Omp A-rEPA) was cloned between the XbaI and EcoRI sites, thus creating the vector pM1816.

The ligation of the PCR fragments and of the digested vector was carried out with the rapid DNA ligation kit from Roche in 5 min at ambient temperature, with a 6× molar excess of fragments. The new vector obtained is named pM1816+ccdA.

In order to remove the kanamycin gene, the plasmid pM1816+ccdA was digested with the ClaI and AscI enzymes. The sticky ends thus freed were treated with mung bean exonuclease (New England BioLabs.) used at 10 U/μl, at 30° C. for 1 h. The religation of the vector at the blunt ends was carried out with the enzyme: T4 DNA ligase New England BioLabs. (400 U/μL) overnight at 16° C., thus deleting 690 by out of 816 by of the kanamycin gene.

CYS21 bacteria (strain sold by Delphi Genetics, having the genotype: F-Cmr mcrA endA1 Δ(mrr-hsdRMS-mcrBC. (restriction-modification), Φ80lacZ, ΔM 15, ΔlacX74, recA1,deoR, Δ(ara,leu)7697, galU, galK, nupG, rpsI, ccdB+ (containing the ccdB gene in their genome)) are electrotransformed with the ligation product, and then plated out onto an antibiotic-free agar plate.

The clones thus obtained were tested as DNA mini preparations and one clone having the correct digestion profile was prepared as a DNA maxi preparation and then sequenced.

The new vector obtained is named pSP1. A schematic representation of this plasmid is given in FIG. 1.

### EXAMPLE 2

### Comparison of the Expression of the rEPA Protein Obtained with the Vectors pSP1, PM1816 and PM1816+ccdA on the Laboratory Scale

The vector/host cell systems tested are described in the table below.

| Vector | Host cell | Agar medium used |
|---|---|---|
| PM1816 | BL21λDE3 | LB + kanamycin 50 μg/ml |
| PM1816 + ccdA | BL21λDE3 | LB + kanamycin 50 μg/ml |
| pSP1 | SE1 | LB |

The SE1 bacteria are derived from BL21λDE3 cells and have the ccdB poison gene in their genome. They are sold by the company Eurogentec and are characterized by the following genotype: F−, CmR, ompT, hsdSB (restriction-, modification), gal, dcm, DE3(lacI, T7 polymerase under the control of the PlacUV5 promoter) Ion, ccdB+.

The BL21λDE3 cells, derived from E. coli B cells, are sold by Invitrogen and are characterized by the following genotype: F⁻ ompT hsdS_{B} (r_{B}⁻m_{B}⁻) gal dcm(DE3).

The LB medium is commercially available from Invitrogen. It comprises, per liter of medium, 10 g of tryptone, 5 g of yeast extract, 10 g of NaCl and qs 1L with H₂O buffered at pH 7.5 by the addition of 5N NaOH.

The prokaryotic cells were transformed by electroporation according to the conditions below:

The E. coli BL21λ DE3 cells are incubated for 30 min in ice after the addition of 8 μL of product derived from the ligation. The transformation is carried out by heat shock for 45 seconds at 42° C.

The transformed bacteria are taken up in SOC medium (comprising, per liter of medium, 20 g of tryptone, 5 g of yeast extract, 0.5 g of NaCl, 10 ml of 250 mM KCl solution, 5 ml of 2M MgCl₂ solution, 20 ml of a 1M glucose solution and qs 1L of H₂O at pH 7 with 5N NaOH) and then incubated for 1 h at 37° C. and plated out on LB agar medium +50 μg/mI kanamycin, before overnight incubation at 37° C.

The SE1 bacteria are incubated for 10 min in ice after the addition of 2 μL of the product derived from the ligation. The transformation by electroporation is carried out using the Gene pulser™ machine (Biorad) in a cuvette comprising 2 electrodes 1 mm apart, under a current of 1.7 kV. The transformed bacteria are taken up in SOC medium and then plated out on antibiotic-free LB agar medium, before overnight incubation at 37° C.

An expression assay was carried out under the following conditions:

The preculture is prepared by inoculating 5 ml of LB medium with a colony picked from a Petri dish onto which the transformed bacteria were plated out, and incubating overnight at 37° C. with shaking. The following day, 50 ml of LB or LB+ kanamycin medium are inoculated with 500 μl of said preculture. This culture is shaken at 37° C., until the OD at 600 nm is between 0.4 and 0.8. At this stage, the culture is divided up into 2 times 25 ml in 2 Erlenmeyer flasks. A solution of IPTG is added to one Erlenmeyer flask at a final concentration of 0.01 M. The other Erlenmeyer flask will serve as a noninduced control.

The two cultures are again shaken for 4 h at 37° C. The OD is then measured, and a 1 ml sample is taken from the 2 Erlenmeyer flasks.

The samples derived from the expression assays in E. coli were taken up in 2× denaturing blue (100 mM Tris-HCl pH 6.8; 20% glycerol; 4% SOS; 0.2% bromophenol blue; 200 mM β-mercaptoethanol) in order to concentrate to an OD of 10 per ml. 20 μl were then loaded onto a 4-20% Tris-glycine gel (PAGEr® Duramide® Precast Gels, Cambrex). The Invitrogen marker 10748-010 was used as molecular weight marker.

After the loading, the gel is subjected to migration for 2 hours in migration buffer (250 mM Tris-HCl; 1.92M glycine; 1% SDS) at a voltage of 175V. The gel is then either visualized with Coomassie blue (rinsing for 30 min in distilled water, staining for 30 min in

Coomassie blue, destaining in a mixture of acetic acid (10%), methanol (30%) and distilled water (60%)), or plotted onto a nitrocellulose membrane by semi-dry electroblotting (2117 Multiphorll Electrophoresis Unit, LKB Bromma) for 2 hours at 65 mA.

After electroblotting, the Western blotting membrane is incubated for 1 hour in a solution of PBS-3% milk. The membrane is then washed three times in PBS-0.05% Tween before being incubated for 1 hour at ambient temperature with the first specific antibody corresponding to an anti-rEPA rabbit serum. The membrane is then washed three times in PBS-0.05% Tween and then incubated for 1 hour at ambient temperature with an HRP-conjugated anti-rabbit IgG goat IgG (Zimed ref 65-6120) diluted to 1/1000 in PBS. After 3 washes in PBS-0.05% Tween, the membrane is incubated for 15 min with the peroxidase substrate (“peroxidase Opti-4CN substrate kit”, Biorad) and rinsed with distilled water.

The results obtained can be summarized in the following way:

The amount of rEPA produced with the pSP1 system in the SE1 bacteria is evaluated at 12% (of total proteins) and that produced with the conventional PM1816 system with kanamycin selection is 14% of total proteins, i.e. amounts that are entirely equivalent. A strongly revealed band at approximately 67 kd corresponding to the rEPA protein is noted, even more strongly in the “induced” samples.

There is no visible difference under these conditions between the conventional PM1816 system and the pSP1 system.

In order to analyze the stability of the plasmid pSP1 in the SE1 bacteria, after culturing for 4 h post-induction, the bacteria are plated out on LB agar medium. 88 clones thus obtained were subsequently analyzed as DNA minipreparations.

All the clones having grown in liquid medium possess the plasmid.

It can thus be concluded that the ccdA gene is functional, since the SE1 bacteria cannot survive without the ccdA antidote gene contained in the plasmid, and that the stability of the plasmid during the expression assay is very good.

### EXAMPLE 3

### Expression of the rEPA Protein in 1-Liter and 30-Liter Fermenters

The objective of this study is to evaluate the vector according to the invention for the production of recombinant proteins on the pilot scale.

### Production of an Experimental Seed Lot (Glycerol Stock)

After transformation of the SE1 strain (Eurogentec) with the plasmid pSp1 as is described in the previous example, the expression level is evaluated on a small scale.

50 ml of LB medium are inoculated at 1:100 with the SE1 cells containing in the plasmid pSp1, and incubated overnight at 37° C.

When the OD (600 nm) reaches the value of 0.8, the IPTG (1 mM) is added for the induction.

After incubation for 4 h, 1 ml of bacterial suspension is removed and centrifuged and the pellet is resuspended in 75 μl of 50 mM Tris buffer, pH 7.4, 1 mM EDTA and 20% sucrose. The volume is then adjusted to 750 μl with 50 mM Tris-HCl buffer, pH 7.4, 1 mM EDTA. The sample is loaded onto a polyacrylamide gel. After staining with coomassie blue, the protein bands are scanned with a GS-710 densitometer from Biorad. Under these conditions, it is observed that the amount of protein of interest represents 12% of the total proteins.

After evaluation of the expression on a small scale, an experimental seed lot comprising 45 vials was prepared according to the following procedure: the clone evaluated is plated out altogether on trypcase-soy agar and incubated at 37° C. for 24 h. The bacterial layer is then taken up with 2 ml of 2×LB medium so as to inoculate 100 ml of modified 2×LB medium in a 500 ml Erlenmeyer flask in order to obtain an initial OD 600 nm of between 0.1 and 0.2 unit. The Erlenmeyer is shaken at 175 rpm at 37° C. until an OD 600 of between 3 and 5 is obtained. The suspension is cooled and then centrifuged at 4000 rpm for 30 min at 4° C. The supernatant is removed and the pellet is taken up with 50 ml of pre-cooled freezing medium. The suspension is then dispensed into nunc cryotubes at a rate of 1 ml per tube, and then stored at a temperature ≦−70° C. in the presence of glycerol.

The modified 2×LB preculture medium contains, per 1 l of mixture: 30 g of yeast extract; 2 ml of 1M MgCl₂, 10 g of NaCl, 10 ml of 2M Tris, pH 7.5±0.1, qs with UF water. It is heat-sterilized (45 min/121° C.: liquid cycle).

The freezing medium is composed of 50% of modified 2×LB medium and 50% of a freezing solution. The freezing solution contains, per liter of mixture, 10 ml of 1M MgSO₄, 200 g of 100% glycerol and 5 g of NaCl, qs with UF water. It is sterilized by 0.22 μm filtration.

### Evaluation in a Fermenter

500 ml of GluSKYE fermentation medium (contains, per 1 L: 40 g of yeast extract, 3 ml of 1M MgCl₂, 0.53 g of K₂SO₄, 1 g of NaCl, 0.84 g of K₂HPO₄, 44 g of glucose, qs with UF water (30 min/121° C.)) are inoculated with 25 ml of a preculture shaken at 175 rpm for 16 h and maintained at a temperature of 37° C. This preculture containing the 2×LB medium was inoculated beforehand with 500 μl of a seed lot vial, stored at −70° C. and containing the producer bacterium in the form of frozen material.

The fermentation equipment used is the Biostat Q (Sartorius), which comprises four 1 L (500 ml working volume) tanks connected to a regulating cabinet and to a supervision system which records and archives the data. The fermentation parameters are the following: pH 7.0; temperature 37° C., initial shaking of 200 rpm, initial aeration of 0.5 L of air/min, pO₂ maintained at 30%. The induction of the r-EPA protein is carried out by addition of 1 mM IPTG when the OD at 600 nm is between 25 and 35 units. The culture is subsequently stopped after 3 h of induction.

A variant of the method consists in inducing the rEPA synthesis at an earlier stage of the culture (between 1 and 3 OD units instead of 25-35), in order to test the stability of the plasmid under more selective conditions. In the same context, another variant consists in prolonging the duration of induction to 20 h.

A first evaluation was carried out in Biostat Q fermenters (BBI) configured as 4 tanks having a working volume of 0.5 liter, which can be individually parametered with respect to pH, pO₂ and temperature. The composition of the medium and also the value of the physical parameters are identical to those selected for the method based on the use of kanamycin. Furthermore, 4 cultures are carried out; the control strain E. coli BL21/pM1816 (Km system) and the strain of the new system E. coil SE1/pSP1 are tested under 2 induction conditions (rapid induction and late induction).

The evaluation on a larger scale was carried out using an Applikon 30L fermenter, adhering to the same principles.

Samples were taken regularly in order to document the following parameters: OD (600 nm), cellular dry weight CDW (g/l), count (CFU/ml), plasmid containment (%), amount of rEPA (PAGE, ELISA).

Each culture sample is treated by osmotic shock so as to release the product present in the periplasm. Each extract is then analyzed by PAGE.

### Monitoring of the Stability of the Plasmid:

The monitoring is carried out by individual analysis of the plasmid content of each colony. Starting from culture samples diluted and deposited on a dish, after incubation, about a hundred colonies are taken up individually in microplates (96 wells) containing LB medium. The plate is then placed in an automated plasmid extraction system (Qiagen bioroBot® Systems) for incubation for 24 h so to allow amplification of the bacteria. They are subsequently centrifuged, lysed and filtered, and the samples are then loaded onto an agarose gel for electrophoresis. The presence of plasmid on the gel is verified after migration and staining.

In the case of the PM1816 system, 100 individual colonies are subcultured on LB agar medium supplemented with kanamycin at a final concentration of 50 μg/ml.

The gel of FIG. 12 documents the presence of rEPA undergoing induction.

With the new system, the induction can be maintained for 24 h without significant loss of plasmid, even if the induction with IPTG is carried out at the beginning of culturing. Under these same rapid induction conditions, only 5% of the cells retain the plasmid with the system based on Km selection, as shown in the table below:

| | Induction at A₆₀₀ = 1 | | Induction at A₆₀₀ = 25 | |
|---|---|---|---|---|
| | E. coli BL21/ | E. coli SE 1/ | E. coli BL21/ | E. coli SE 1/ |
| | PM1816 | pSP1 | PM1816 | pSP1 |
| A₆₀₀ | 65 | 49 | 41 | 47 |
| % P⁺ | 5% | 100% | 90% | 98% |
| rEPA (mg/liter) | 36 | 350 | 280 | 603 |

Productivity of rEPA in 1 L fermenter after 24 h of culture
% P+ measures the percentage of cells containing the plasmid among the total viable cells.
the amount of rEPA is evaluated by ELISA as is described in the previous examples.

### Evaluation in a 30 l Fermenter:

The new construct was subsequently tested at the standard clinical batch production scale, i.e. 30l. The culture parameters remain the same as those described in the preceding cultures. The volume of culture medium in the fermenter is 20 liters. The rEPA production kinetics are given in FIG. 13.

We evaluated the new vector for the production of a recombinant protein. This system does away with the presence of an antibiotic-resistance gene and therefore no longer has recourse to the use of antibiotics whatever the culture step. The pilot-scale evaluation in a 30 l fermenter showed that the plasmid is maintained throughout the culture, without any loss being observed.

The productivity of protein of interest obtained is greater than that observed with the conventional system using an antibiotic-resistance gene as selectable marker.

### EXAMPLE 4

### Analysis of the Advantage of the Cer Fragment

In order to evaluate the contribution of the Cer fragment to the increase in production observed, various vectors were constructed.

The vector pM1800 was amplified by PCR in order to remove the kanamycin-resistance gene and to create the PstI and Acc65I restriction sites.

The SEQ ID No. 8 cassette was constructed by combined PCRs from the vector pM1800.

First of all, the mob promoter and the 5′ part of the ccdA gene were amplified from the plasmid pStaby (Delphi gentic). The PCR primer provides a fragment of the 3′ part of the kanamycin gene. This PCR is named C1.

A second PCR makes it possible to amplify the 3′ part of the ccdA gene. The PCR primer provides a fragment of the 5′ part of the kanamycin gene. This amplification is named C2.

The third PCR concerns the amplification of the kanamycin gene from pM1800. The 5′ primer provides a fragment of the 5′ part of the ccdA gene. The 3′ primer provides a fragment of the 3′ part of the ccdA gene. This PCR is named C3.

PCR C1 is combined with PCR C2 and the product is subsequently combined with PCR C3. The fragment obtained is digested with the PstI and Acc65I enzymes and cloned into the vector pM1800, digested with the same enzymes.

The ligation of the two fragments was carried out with the enzyme: T4 DNA ligase, New England BioLabs. (400 U/μL), overnight at 16° C. The new vector created is named pSP301. A representation of this vector is given in FIG. 6.

A version of this vector without the cer fragment was constructed. The pSP301 vector was digested with the PacI and AscI enzymes, thus deleting the cer fragment. The sticky ends thus freed were treated with mung bean exonuclease (New England BioLabs) used at 10 U/μl at 30° C. for 1 h. The religation of the vector at the two blunt ends was carried out with the enzyme: T4 DNA ligase, New England BioLabs (400 U/μL) overnight at 16° C.

The new vector thus constructed is named pSP2. A schematic representation of the plasmid pSP2 is given in FIG. 7.

The SEQ ID No. 9 cassette (containing RBS-Omp A-rEPA, 2024 bp), isolated from the plasmid pM1816, was cloned into the pSP301 and pSP2 vectors between the XbaI and EcoRI sites. The kanamycin gene was subsequently excised from these vectors by homologous recombination as described in Example 4. The plasmids obtained are named respectively pSP6 and pSP4, a schematic representation of which is given in FIGS. 8 and 9.

The SE1 strain was transformed with the pSP6 and pSP4 vectors and the BL21ADE3 strain was transformed with the pM1816 vector. A comparative expression assay was carried out using these 3 vectors. The same working conditions as those of Example 2 were used.

The best rEPA production results were obtained with the constructs containing the cer fragment.

In order to reinforce the first results obtained, the same type of study was carried out with a second antigen: the Helicobacter pylori AlpA protein.

The following vectors were constructed.

The AlpA protein gene (SEQ ID No. 4) was cloned into the pSP301 and pSP2 vectors (previously described) between the NcoI/XabaI sites. The kanamycin gene of these new plasmids is excised by homologous recombination, as described in Example 4, thus creating respectively the pSP5 and pSP3 vectors. A schematic representation of these vectors is given in FIGS. 10 and 11.

The SE1 strain was transformed with the pSP5 and pSP3 vectors and the BL21λDE3 strain was transformed with the pMH.P3.1 vector. A comparative expression assay was carried out using these 3 vectors. The same working conditions as those of Example 2 were used.

The results obtained show that the amount of protein of interest produced (% expressed relative to the total proteins) is greater with the “no antibiotic” vectors containing the Cer sequence. The densitometric analysis of the bands obtained by SDS PAGE electrophoresis clearly indicates a decrease in expression in the absence of the Cer fragment.

The comparison of the various constructs shows that a “no antibiotic” +cer vector results in productivities that are much higher than those obtained with an “antibiotic” +cer vector, which results in a greater productivity than that obtained with an “antibiotic” vector without Cer.

Furthermore, it is observed that the “no antibiotic” +cer vector results in an increase in production of the protein of interest of at least 10% compared with an “antibiotic” system without cer.

### EXAMPLE 4

### Deletion of the Kanamycin Gene by Homologous Recombination

The pSP301 or pSP2 vectors are digested with the ClaI enzyme.

2 μg of plasmid DNA are digested with 6U of ClaI enzyme, for 1 h at 37° C. 100 μl of electrocompetent CYS21 bacteria (Delphi Genetics) are then transformed in 200 ng of this digestion. The entire transformation is plated out onto an LB plate.

The clones obtained are subsequently analyzed as DNA minipreparations by enzymatic digestion.

The analysis of clones obtained after transfection of electrocompetent CYS21 bacteria with the linearized DNA shows that all the colonies obtained contain a recombined plasmid. The sequencing of 2 clones taken randomly made it possible, in addition, to confirm that the assembly of the ccdA gene was in accordance with the expected result.

This homologous combination was carried out with the plasmids pSP3, pSP5 and pSP4, pSP6.

The homologous recombination method provides a positive selection of the recombined clones insofar as the ccdA gene can be functional only after elimination of the kanamycin-resistance gene. We can thus be certain that the transformed bacteria have lost the kanamycin-resistance gene, which is not the case when the system based on an enzyme digestion step followed by religation is used. This method therefore has the effect of completely doing away with any additional analysis aimed at documenting the absence of the kanamycin-resistance gene. This additional analysis will conventionally be carried out on a plasmid DNA preparation analyzed by restriction mapping or alternatively by means of the specific PCR amplification method.

1. A self-replicating vector devoid of any antibiotic-resistance gene, comprising:
a sequence encoding the ccdA protein functionally linked to a first promoter,
the sequence of the Cer locus, and
a heterologous sequence functionally linked to a second promoter.

2. The vector as claimed in claim 1, in which the first promoter is the mob constitutive promoter.

3. The vector as claimed in claim 1, in which the second promoter is an inducible promoter.

4. The vector as claimed in claim 3, in which the second promoter is the T7 promoter.

5. The vector as claimed in claim 1, in which the heterologous sequence encodes a vaccine antigen.

6. The vector as claimed in claim 5, in which the heterologous sequence encodes the rEPA protein.

7. The vector as claimed in claim 1, in which the heterologous sequence encodes a sequence that can be used in the context of gene therapy.

8. The vector as claimed in claim 1, in which the heterologous sequence encodes an enzyme.

9. A prokaryotic cell expressing the ccdB protein, containing an expression vector as defined in claim 1.

10. The prokaryotic cell as claimed in claim 9 corresponding to an E. coli cell.

11. A method for producing a heterologous protein, the method comprising:
(a) inoculating an appropriate culture medium with cells as defined in claim 9,
(b) fermenter culturing the cells thus transformed in the absence of antibiotic, and
(c) recovering the heterologous protein produced during step (b) from the supernatant or from the cell pellet.

12. The method, as claimed in claim 11, for producing a heterogeneous protein in which the heterogeneous protein is rEPA.

13. A method for producing a self-replicating vector as defined in claim 1, the method comprising:
(a) inoculating an appropriate culture medium with prokaryotic cells expressing the ccdB protein and containing a vector as defined above,
(b) fermenter culturing the cell thus transformed in the absence of antibiotic, and
(c) recovering the vector produced during step (b).

14. A method for constructing a self-replicating expression vector as defined in claim 1, the method comprising:
(a) constructing a self-replicating vector comprising an antibiotic-resistance gene flanked respectively by a sequence 1 and a sequence 2, in which the sequences 1 and 2 are two overlapping sequences of the sequence encoding the ccdA protein, which, after homologous recombination, reconstitutes a functional sequence,
(b) linearizing said vector by using a restriction enzyme which cleaves the vector only between the sequences 1 and 2,
(c) transforming a prokaryotic cell expressing the ccdB protein, and
(d) recovering the prokaryotic cells comprising the self-replicating vector.


SEQUENCE LISTING (9 sequences)

SEQ ID NO: 1 - DNA, 284 bp, Escherichia coli
ccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcgtagccatcga

SEQ ID NO: 2 - DNA, 31 bp, Artificial Sequence
  Synthetic; Primer
tccttgcggccgcacatcaccgatggggaag

SEQ ID NO: 3 - DNA, 26 bp, Artificial Sequence
  Synthetic; Primer
ctgaattctcaccagtccctgttctc

SEQ ID NO: 4 - DNA, 1512 bp, Helicobacter pylori
atggctagcataagttatgccgaagatgatggagggtttttcaccgtcggttatcagctcgggcaggtcatgcaagatgtccaaaacccaggcggcgctaaaagcgacgaactcgccagagagcttaacgctgatgtaacgaacaacattttaaacaacaacaccggaggcaatgtcgcaggggcgttgagtaacgctttctcccaatacctttattcgcttttaggggcttacccaacaaaactcaatggtagcgatgtgtctgcgaacgctcttttaagtggtgcggtaggcagtgggacttgcacggctgcagggacggccggtggcacatctcttaacactcaaagcgcttgcaccgctgcgggatattactggctccctagcttgactgacaggattttaagcacgatcggcagccagactaactacggcacgaacaccaatttccccaacatgcaacaacagctcacctacttgaatgcggggaatgtgttttttaatgcgatgaataaggctttagggaagaatgggactgctactactagtggcactagcagtgcaactggttcagatggtcaaacttactccacacaagccatccaataccttcaaaaccaacaaaatatcttaaataacgcagcgaacttgctcaagcaagatgaattgctcctagaagctttcaactctgccgtagctgctaacattgggaataaggaattcaattcagccgcttttacaggtttggtgcaaggcattattgatcaatctcaattggtttataacgagctcactaaaaacaccattagcgggagcgcggttaatggcgctgggataaactccaaccaagctaatgctgtgcaagggcgtgccagtcagctccctaacgctctttataacgcgcaagtaactttggataaaatcaatgcgctcaataatcaggtgagaagcatgccttacttgccccaattcagagccgggaacagccgtgcaacgaatattttaaacgggttttacaccaaaataggctataagcaattcttcgggaagaaaaggaatatcggtttgcgctattatggtttcttttcttataacggagcgagcgtgggctttagatccactcaaaataatgtagggttatacacttatggggtggggactgatgtgttgtataacatctttagccgctcctatcaaaaccgctctgtggatatgggcttttttagcggcatccaattagccggtgagactttccaatccacgctcagagatgaccccaatgtgaaattgcatgggaaaatcaataacacgcacttccagttcctctttgacttcggtatgaggatgaacttcggtaagttggacgggaaatccaaccgccacaaccagcacacggtggaatttggcgtagtggtgcctacgatttataacacttattacaaatcagcagggactaccgtgaagtatttccgtccttatagcgtttattggtcttatgggtattcattctaa

SEQ ID NO: 5 - DNA, 1184 bp, Influenza virus
atgaaagcaaaactactggtcctgttatgcacatttacagctacatatgcagacacaatatgtataggctaccatgccaacaactcaaccgacactgttgacacagtacttgagaagaatgtgacagtgacacactctgtcaacctgcttgaggacagtcacaatggaaaactatgtctattaaaaggaatagccccactacaattgggtaattgcagcgttgccgggtggatcttaggaaacccagaatgcgaattactgatttccaaggaatcatggtcctacattgtagaaaaaccaaatcctgagaatggaacatgttacccagggtatttcgccgactatgaggaactgagggagcaattgagttcagtatcttcatttgagaggttcgaaatattccccaaagaaagctcatggcccaaccacaccgtaaccggagtatcagcatcatgctcccataatgggaaaagcagtttttacaaaaatttgctatggctgacggggaagaatggtttgtacccaaacctgagcaagtcctatgcaaacaacaaagagaaagaagtccttgtactatggggtgttcatcacccgcctaacataggtgaccaaagggccctctatcatacagaaaatgcttatgtctctgtagtgtcttcacattatagcagaaaattcaccccagaaatagccaaaagacccaaagtaagagatcaagaaggaagaatcaactactactggactctgcttgaacccggggatacaataatatttgaggcaaatggaaatctaatagcgccaagatatgctttcgcactgagtagaggctttggatcaggaatcatcacctcaaatgcaccaatggatgaatgtgatgcgaagtgccaaacacctcagggagctataaacagcagtcttcctttccagaatgtacacccagtcacaataggagagtgtccaaagtatgtcaggagtgcaaaattaaggatggttacaggactaaggaacatcccatccattcaatccagaggtttgtttggagccattgccggtttcattgaaggggggtggactggaatggtagatggttggtatggttatcatcatcagaatgagcaaggctctggctatgctgcagatcaaaaaagcacacaaaatgccattaatgggattacaaacaagtg

SEQ ID NO: 6 - DNA, 4443 bp, Plasmodium falciparum
atgcagaggtcgcctctggaaaaggccagcgttgtctccaaactttttttcagctggaccagaccaattttgaggaaaggatacagacagcgcctggaattgtcagacatataccaaatcccttctgttgattctgctgacaatctatctgaaaaattggaaagagaatgggatagagagctggcttcaaagaaaaatcctaaactcattaatgcccttcggcgatgttttttctggagatttatgttctatggaatctttttatatttaggggaagtcaccaaagcagtacagcctctcttactgggaagaatcatagcttcctatgacccggataacaaggaggaacgctctatcgcgatttatctaggcataggcttatgccttctctttattgtgaggacactgctcctacacccagccatttttggccttcatcacattggaatgcagatgagaatagctatgtttagtttgatttataagaagactttaaagctgtcaagccgtgttctagataaaataagtattggacaacttgttagtctcctttccaacaacctgaacaaatttgatgaaggacttgcattggcacatttcgtgtggatcgctcctttgcaagtggcactcctcatggggctaatctgggagttgttacaggcgtctgccttctgtggacttggtttcctgatagtccttgccctttttcaggctgggctagggagaatgatgatgaagtacagagatcagagagctgggaagatcagtgaaagacttgtgattacctcagaaatgattgaaaatatccaatctgttaaggcatactgctgggaagaagcaatggaaaaaatgattgaaaacttaagacaaacagaactgaaactgactcggaaggcagcctatgtgagatacttcaatagctcagccttcttcttctcagggttctttgtggtgtttttatctgtgcttccctatgcactaatcaaaggaatcatcctccggaaaatattcaccaccatctcattctgcattgttctgcgcatggcggtcactcggcaatttccctgggctgtacaaacatggtatgactctcttggagcaataaacaaaatacaggatttcttacaaaagcaagaatataagacattggaatataacttaacgactacagaagtagtgatggagaatgtaacagccttctgggaggagggatttggggaattatttgagaaagcaaaacaaaacaataacaatagaaaaacttctaatggtgatgacagcctcttcttcagtaatttctcacttcttggtactcctgtcctgaaagatattaatttcaagatagaaagaggacagttgttggcggttgctggatccactggagcaggcaagacttcacttctaatggtgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggacatctccaagtttgcagagaaagacaatatagttcttggagaaggtggaatcacactgagtggaggtcaacgagcaagaatttctttagcaagagcagtatacaaagatgctgatttgtatttattagactctccttttggatacctagatgttttaacagaaaaagaaatatttgaaagctgtgtctgtaaactgatggctaacaaaactaggattttggtcacttctaaaatggaacatttaaagaaagctgacaaaatattaattttgcatgaaggtagcagctatttttatgggacattttcagaactccaaaatctacagccagactttagctcaaaactcatgggatgtgattctttcgaccaatttagtgcagaaagaagaaattcaatcctaactgagaccttacaccgtttctcattagaaggagatgctcctgtctcctggacagaaacaaaaaaacaatcttttaaacagactggagagtttggggaaaaaaggaagaattctattctcaatccaatcaactctatacgaaaattttccattgtgcaaaagactcccttacaaatgaatggcatcgaagaggattctgatgagcctttagagagaaggctgtccttagtaccagattctgagcagggagaggcgatactgcctcgcatcagcgtgatcagcactggccccacgcttcaggcacgaaggaggcagtctgtcctgaacctgatgacacactcagttaaccaaggtcagaacattcaccgaaagacaacagcatccacacgaaaagtgtcactggcccctcaggcaaacttgactgaactggatatatattcaagaaggttatctcaagaaactggcttggaaataagtgaagaaattaacgaagaagacttaaaggagtgcttttttgatgatatggagagcataccagcagtgactacatggaacacataccttcgatatattactgtccacaagagcttaatttttgtgctaatttggtgcttagtaatttttctggcagaggtggctgcttctttggttgtgctgtggctccttggaaacactcctcttcaagacaaagggaatagtactcatagtagaaataacagctatgcagtgattatcaccagcaccagttcgtattatgtgttttacatttacgtgggagtagccgacactttgcttgctatgggattcttcagaggtctaccactggtgcatactctaatcacagtgtcgaaaattttacaccacaaaatgttacattctgttcttcaagcacctatgtcaaccctcaacacgttgaaagcaggtgggattcttaatagattctccaaagatatagcaattttggatgaccttctgcctcttaccatatttgacttcatccagttgttattaattgtgattggagctatagcagttgtcgcagttttacaaccctacatctttgttgcaacagtgccagtgatagtggcttttattatgttgagagcatatttcctccaaacctcacagcaactcaaacaactggaatctgaaggcaggagtccaattttcactcatcttgttacaagcttaaaaggactatggacacttcgtgccttcggacggcagccttactttgaaactctgttccacaaagctctgaatttacatactgccaactggttcttgtacctgtcaacactgcgctggttccaaatgagaatagaaatgatttttgtcatcttcttcattgctgttaccttcatttccattttaacaacaggagaaggagaaggaagagttggtattatcctgactttagccatgaatatcatgagtacattgcagtgggctgtaaactccagcatagatgtggatagcttgatgcgatctgtgagccgagtctttaagttcattgacatgccaacagaaggtaaacctaccaagtcaaccaaaccatacaagaatggccaactctcgaaagttatgattattgagaattcacacgtgaagaaagatgacatctggccctcagggggccaaatgactgtcaaagatctcacagcaaaatacacagaaggtggaaatgccatattagagaacatttccttctcaataagtcctggccagagggtgggcctcttgggaagaactggatcagggaagagtactttgttatcagcttttttgagactactgaacactgaaggagaaatccagatcgatggtgtgtcttgggattcaataactttgcaacagtggaggaaagcctttggagtgataccacagaaagtatttattttttctggaacatttagaaaaaacttggatccctatgaacagtggagtgatcaagaaatatggaaagttgcagatgaggttgggctcagatctgtgatagaacagtttcctgggaagcttgactttgtccttgtggatgggggctgtgtcctaagccatggccacaagcagttgatgtgcttggctagatctgttctcagtaaggcgaagatcttgctgcttgatgaacccagtgctcatttggatccagtaacataccaaataattagaagaactctaaaacaagcatttgctgattgcacagtaattctctgtgaacacaggatagaagcaatgctggaatgccaacaatttttggtcatagaagagaacaaagtgcggcagtacgattccatccagaaactgctgaacgagaggagcctcttccggcaagccatcagcccctccgacagggtgaagctctttccccaccggaactcaagcaagtgcaagtctaagccccagattgctgctctgaaagaggagacagaagaagaggtgcaagatacaaggctttag

SEQ ID NO: 7 - DNA, 357 bp, Artificial Sequence
  Synthetic
cgccgcagccgccgtaggcggtcacgactttgcgaagcaaagtctagtgagtatactcaagcattgagtggcccgccggaggcaccgccttgcgctgcccccgtcgagccggttggtcaccaaaagggaggggcaggcatgaagcagcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtga

SEQ ID NO: 8 - DNA, 1463 bp, Artificial Sequence
  Synthetic; 5′ccdA-KANA-3′ccdA
ctgcagcgccgcagccgccgtaggcggtcacgactttgcgaagcaaagtctagtgagtatactcaagcattgagtggcccgccggaggcaccgccttgcgctgcccccgtcgagccggttggtcaccaaaagggaggggcaggcatgaagcagcgtattacagtgacagttgacagcgacagctatcagttgctcaaggcatatgatgtcaatatctccggtctggtaagcacaaccatgcagaatgaagcccgtcgtctgcgtgccgaacgggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgtgcgtattacagtgacagttgacagcgacagctatcagttgctcaaggcatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgagcatgcaccattccttgcggcggcggtgctcaacggcctcaacctactacggtacc

SEQ ID NO: 9 - DNA, 2030 bp, Artificial Sequence
  Synthetic; RBS-ompA-rEPA
tctagataacgagggcaaaaaatgaaaaagacagctatcgcgattgcagtggcactggctggtttcgctaccgtagcgcaggccgcgaatttggccgaagaagctttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcgtgaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaactgccgcgaccggccggctcccttcgcaggagccggccttctcggggcctggccatacatcaggttttcctgatgccagcccaatcgaatatgaattc